(19) United States
(12) Patent Application Publication
(10) Pub. No.: US 2023/0182991 A1
(43) Pub. Date: Jun. 15, 2023

(54) DEVICE FOR AUTOMATICALLY TAKING OUT ONE OF MEDICINE APPLICATOR STICKS FOR USE

(71) Applicant: HUANGHUA PROMISEE DENTAL CO., LTD., Huanghua, Hebei (CN)

(72) Inventor: Qingxuan He, Huanghua, Hebei (CN)

(73) Assignee: HUANGHUA PROMISEE DENTAL CO., LTD., Huanghua, Hebei (CN)

(21) Appl. No.: 17/795,480

(22) PCT Filed: Jan. 18, 2021
(86) PCT No.: PCT/CN2021/072441

§ 371 Date: Jul. 26, 2022

(30) Foreign Application Priority Data
Jul. 17, 2020 (CN) 202010689451.6

(51) Int. Cl. B65D 83/02; A61L 2/10; A61L 2/24; A61L 2/26

(52) U.S. Cl. CPC B65D 83/02; A61L 2/10; A61L 2/24; A61L 2/26; A61L 2202/11; A61L 2202/14; A61L 2202/24

(57) ABSTRACT

A device for automatically taking a medicine applicator stick is provided, which includes a box body and a medicine applicator stick bin, a lifting mechanism, a disinfection mechanism and a controller which are provided within the box body. The medicine applicator stick bin is used for accommodating medicine applicator sticks and can slide down one of the medicine applicator sticks each time; the medicine applicator stick bin is joined to an ejector fork of the lifting mechanism via an inclined sliding track on the box body. The lifting mechanism can drive the medicine applicator stick to rise; the disinfection mechanism corresponds to a dipping end of the medicine applicator stick and can disinfect the dipping end of the medicine applicator stick of the medicine applicator stick; and the lifting mechanism and the disinfection mechanism are each electrically connected to the controller.

### CROSS REFERENCE TO RELATED APPLICATION

This patent application claims the benefit and priority of Chinese Patent Application No. 202010689451.6, entitled “DEVICE FOR AUTOMATICALLY TAKING OUT ONE OF MEDICINE APPLICATOR STICKS FOR USE” filed on Jul. 17, 2020, the disclosure of which is incorporated by reference herein in its entirety as part of the present application.

### TECHNICAL FIELD

The present disclosure relates to the technical field of medical equipment, and in particular, to a device for automatically taking out one of medicine applicator sticks for use.

### BACKGROUND ART

During dental treatment, some materials, such as an acid etching agent, a binder, a hole lining, and a hemostatic agent, often need to be coated on the affected parts of the teeth to meet the treatment requirements. During the application, depending on the types and the usage amount of medicament, a dental medicine applicator stick is often selected.

There are many devices for accommodating dental medicine applicator sticks, such as a plastic bag, a plastic box, and a plastic bucket. The medicine applicator sticks can be taken out by means of directly opening and rotating covers of these devices, as well as pressing material taking components of these devices and the like by people's hands during use. During taking out the medicine applicator stick, the medicine applicator stick is extremely prone to be partially or completely exposed to the air, thereby greatly increasing the possibility of the medicine applicator stick being contaminated.

### SUMMARY

The objective of some embodiments is to provide a device for automatically taking out one of medicine applicator sticks for use so as to solve the problem existing in the prior art. So, the hygiene of the operation of taking out the medicine applicator stick is improved, and the risk of contamination of the medicine applicator stick is reduced.

In order to achieve the above objective, the present disclosure provides the following solution.

The present disclosure provides a device for automatically taking a medicine applicator stick, including a box body and a medicine applicator stick bin, a lifting mechanism, a disinfection mechanism and a controller which are provided in the box body, wherein the medicine applicator stick bin is configured for accommodating the medicine applicator sticks and capable of sliding down the one of the medicine applicator sticks at a time, and the medicine applicator stick bin is jointed to an ejector fork of the lifting mechanism via an inclined sliding track of the box body, the lifting mechanism is capable of driving the one of the medicine applicator sticks to rise, the disinfection mechanism is arranged to correspond to a dipping end of the one of the medicine applicator sticks and is capable of disinfecting the dipping end of the one of the medicine applicator sticks, the lifting mechanism and the disinfection mechanism are each electrically connected to the controller.

Preferably, a bin cover of the medicine applicator stick bin is snap-fitted with a bin body of the medicine applicator stick bin, the bin body is snap-fitted with the box body, a bottom of the medicine applicator stick bin is an inclined surface; a discharge port is provided on a bin wall, which intersects with the inclined surface, of the medicine applicator stick bin; and a sliding cover is slidably provided on the discharge port.

Preferably, a side of the medicine applicator stick bin is provided with an access window; a side wall of the access window is provided with bosses which are configured to abut against a bottom of the sliding cover; the access window is hollow and a top cover is rotatably provided at a top of the access window; and the top cover is flush with the bin cover.

Preferably, the disinfection mechanism includes an ultraviolet lamp and a grating sensor, the ultraviolet lamp and the grating sensor are each electrically connected to the controller, and the ultraviolet lamp is arranged at a bottom of the access window and corresponds to the dipping end of the one of the medicine applicator sticks.

Preferably, the ultraviolet lamp is arranged in the box body, a fixing plate is arranged in the box body, a fixing plate is arranged in the box body, the grating sensor is provided on a middle of the fixing plate; a top of the fixing plate is provided with a light sensor which is electrically connected to a switch of the controller.

Preferably, the lifting mechanism includes an electric motor, a lifting frame, a gear and a rack, a rotating shaft of the electric motor is connected to the gear, the rack is provided on the lifting frame, the gear is meshed with the rack, the ejector fork is provided at a top of the lifting frame, vertical blocking bars are provided on sides of the lifting frame, and each of the blocking bars is provided with a positioning notch, the blocking bars match with the grating sensor.

Preferably, the rotating shaft of the electric motor is connected to the gear via a speed reducer.

Preferably, a bottom of the box body is provided with a base which is provided with a power supply cabin for accommodating a power supply, an electric quantity indicator light and a charging port.

Preferably, the base is provided with an anti-inclining means.

Some embodiments achieve the following technical effects with respect the prior art.

The device for automatically taking out one of medicine applicator sticks for use in some embodiments has multiple dustproof, exposure-proof, and touch-proof function, thereby reducing the risk of contamination during the process of taking out the medicine applicator stick for use due to touching, exposure, and splashing of foreign matters and the like. Also, the device can perform the disinfection by using ultraviolet, and facilitate to automatically take out the medicine applicator stick, thus improving the hygiene of the medicine applicator stick during the operation of taking out the medicine applicator stick for use.

### BRIEF DESCRIPTION OF THE DRAWINGS

To describe the technical solutions in the embodiments of the present disclosure or in the prior art more clearly, the following briefly introduces the accompanying drawings required for describing the embodiments. Apparently, the accompanying drawings in the following description show merely some embodiments of the present disclosure, and a person of ordinary skill in the art may still derive other drawings from these accompanying drawings without creative efforts.

FIG. 1 is a structural schematic diagram of a device for automatically taking out one of medicine applicator sticks for use according to an embodiment of the present disclosure;

FIG. 2 is another structural schematic diagram of the device for automatically taking out one of medicine applicator sticks according to an embodiment of the present disclosure;

FIG. 3 is a partial structural schematic diagram of the device for automatically taking out one of medicine applicator sticks according to an embodiment of the present disclosure;

FIG. 4 is another partial structural schematic diagram of the device for automatically taking out one of medicine applicator sticks according to an embodiment of the present disclosure;

FIG. 5 is a structural schematic diagram of a medicine applicator stick bin of the device for automatically taking out one of medicine applicator sticks according to an embodiment of the present disclosure;

FIG. 6 is a structural schematic diagram of an access window of the device for automatically taking out one of medicine applicator sticks according to an embodiment of the present disclosure;

In the figures: 1 medicine applicator stick bin; 2 discharge port; 3 sliding cover; 4 access window; 5 top cover; 6 boss; 7 fixing plate; 8 ultraviolet lamp; 9 grating sensor; 10 light sensor; 11 controller; 12 electric motor; 13 speed reducer; 14 lifting frame; 15 gear; 16 rack; 17 base; 18 power supply cabin; 19 box body; 20 inclined sliding track; 21 anti-inclining means.

### DETAILED DESCRIPTION OF THE EMBODIMENTS

The technical solutions in the embodiments of the present disclosure will be clearly and completely described below in conjunction with the accompanying drawings in the embodiments of the present disclosure. Apparently, the described embodiments are only a part of the embodiments of the present disclosure, rather than all the embodiments. Based on the embodiments of the present disclosure, all other embodiments obtained by those of ordinary skill in the art without creative work shall fall within the protection scope of the present disclosure.

The objective of some embodiments is to provide a device for automatically taking out one of medicine applicator sticks for use so as to solve the problem existing in the prior art. So, the hygiene of the operation of taking out the medicine applicator stick is improved, and the risk of contamination of the medicine applicator stick is reduced.

In order to make the objectives, features, and advantages mentioned above of the present disclosure more apparent and easily understood, the present disclosure will be further described in detail below with reference to the drawings and particular implementations.

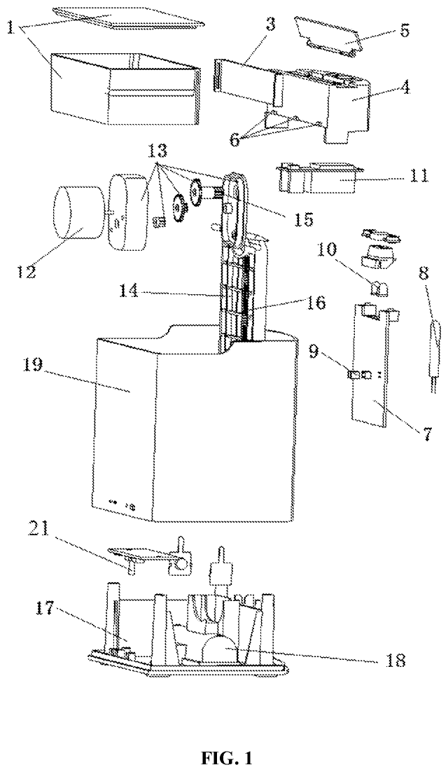

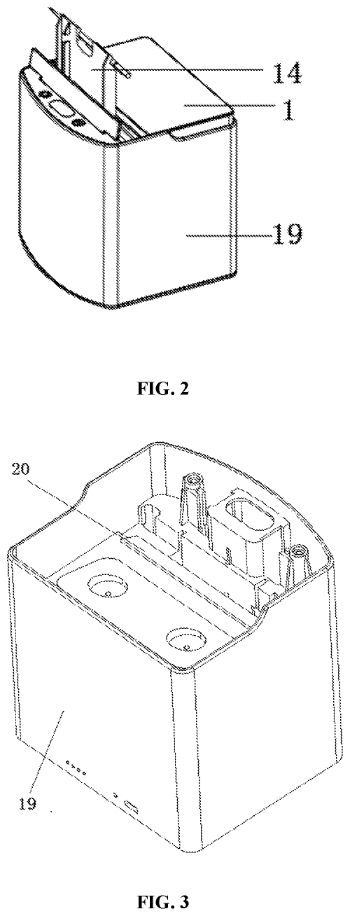

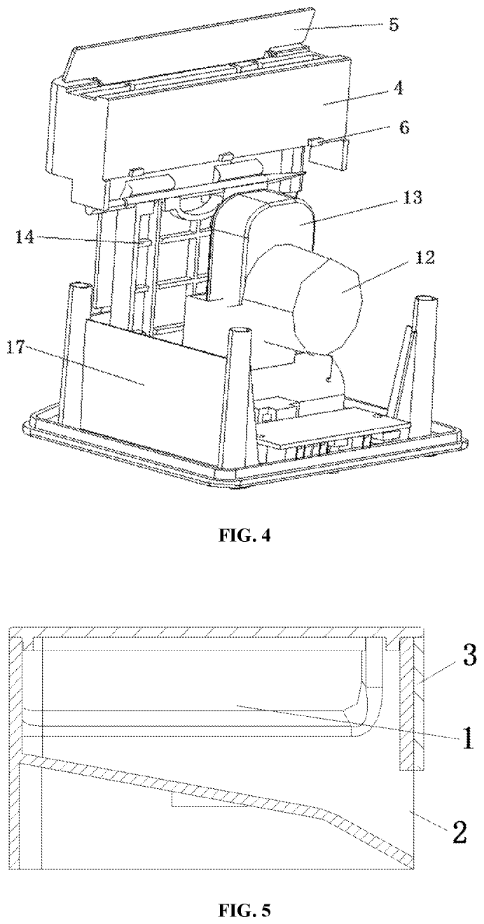

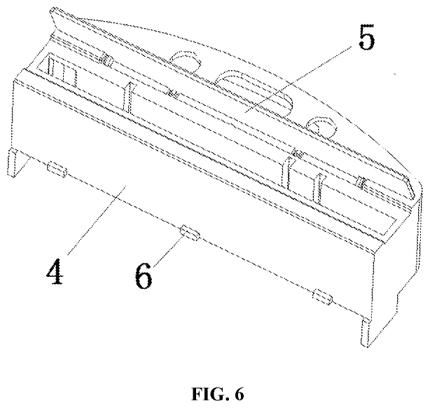

As shown in FIGS. 1-6, this embodiment provides a device for automatically taking out one of medicine applicator sticks. The device includes a box body 19 and a medicine applicator stick bin 1, a lifting mechanism, a disinfection mechanism and a controller 11 which are provided in the box body 19. The medicine applicator stick bin 1 is configured for accommodating the medicine applicator sticks and capable of sliding down the one of the medicine applicator sticks at a time. The medicine applicator stick bin 1 is jointed to an ejector fork of the lifting mechanism via an inclined sliding track 20 of the box body 19. The lifting mechanism is capable of driving the one of the medicine applicator sticks to rise. The disinfection mechanism is arranged to correspond to the dipping end of the one of the medicine applicator stick and is capable of disinfecting the dipping end of the one of the medicine applicator sticks. The lifting mechanism and the disinfection mechanism are each electrically connected to the controller 11.

A bin cover of the medicine applicator stick bin 1 is snap-fitted with the bin body of the medicine applicator stick bin 1, the bin body is snap-fitted with the box body 19. The bottom of the medicine applicator stick bin 1 is an inclined surface. A discharge port 2 is provided on a bin wall, which intersects with the inclined surface, of the medicine applicator stick bin 1. A sliding cover 3 is slidably provided on the discharge port 2, so that the sliding cover 3 is vertically slidably on the discharge port 2.

A side of the medicine applicator stick bin 1 is provided with an access window 4. A side wall of the access window 4 is provided with bosses 6 which are configured to abut against a bottom of the sliding cover 3. The access window 4 is hollow and a top cover 5 is rotatably provided on the top of the access window 4. The top cover 5 can be opened by the ejector fork. The top cover 5 is flush with the bin cover, which makes appearance of the device to be aesthetic and in alignment.

The disinfection mechanism includes an ultraviolet lamp 8 and a grating sensor 9, where the ultraviolet lamp 8 and the grating sensor 9 are each electrically connected to the controller 11. And the ultraviolet lamp 8 is arranged at the bottom of the access window 4 and to correspond to the dipping end of one of the medicine applicator sticks. The bottom of the ultraviolet lamp 8 is arranged in the box body 19 by means of a socket connection and is electrically connected to the controller. A fixing plate 7 is arranged in the box body; the middle of the fixing plate 7 is provided with a grating sensor 9, and the top of the fixing plate 7 is provided with a light sensor 10 which is electrically connected to a switch on the controller 11, so as to facilitate the operation of lifting the lifting frame 14 by gesture sensing. And, the controller is further provided with a switch button and a lifting button. A working state prompting light, the switch button and the lifting button are further arranged at the top of the fixing plate 7.

The lifting mechanism includes an electric motor 12, the lifting frame 14, a gear 15 and a rack 16. A rotating shaft of the electric motor 12 is connected to the gear 15; the rack 16 is provided on the lifting frame 14; the gear 15 is meshed with the rack 16; and the ejector fork is provided at the top of the lifting frame 14. A groove on the ejector fork is also inclined and the ejector fork is provided with protrusions, so as to provide smoothly transition between the protrusions and the inclined sliding track 20, and prevent the top of the medicine applicator stick from touching the top cover 5. Vertical blocking bars are provided on sides of the lifting frame 14, and a positioning notch is provided on the blocking bar. And, the blocking bars match with the grating sensor 9, and the blocking bars can slide up and down in a groove of the grating sensor 9. When the positioning notch of the blocking bar moves to the grating sensor 9 (namely, when the lifting frame 14 is located at the lowest position), the grating sensor 9 is activated and transmits a positioning signal to the controller, and the controller controls the ultraviolet lamp 8 to be turned on so as to disinfect the dipping end of the stick. The rotating shaft of the electric motor 12 is connected to the gear 15 via speed reducer 13.

The bottom of the box body 19 is provided with a base 17, and the base 17 is provided with a power supply cabin 18 for accommodating a power supply, an electric quantity indicator light, a charging indicator light and a charging port. An anti-inclining means 21 is arranged on the base 17. The anti-inclining means 21 in this embodiment is a contact switch. When the base 17 is placed flatly, the contact switch is turned on to connect the entire control circuit, and the device works normally. When the base 17 is inclined, the contact switch disconnects the entire control circuit, and the device stops working, thereby preventing the medicine applicator stick from being fallen out due to incline thereof.

When the device of this embodiment is used, the medicine applicator stick bin 1 is first placed in a box body 19, and the medicine applicator stick is automatically slid onto the ejector fork of the lifting frame 14 from the medicine applicator stick bin 1 along the inclined sliding track 20. After the power switch is turned on, the grating sensor 9 senses that the lifting frame 14 is at the lowest position, and the controller 11 controls to turn on the ultraviolet lamp 8, and disinfects the dipping end of the medicine applicator stick with ultraviolet disinfection for 3 s-5 s. When it is desired to take out the medicine applicator stick for use, the electric motor 12 can be started by manually pressing the switch button or by using the light sensor 10 to sense gestures, and the lifting frame 14 is lifted up and extended out of the access window 4. And, the medicine applicator stick is delivered out of the access window by the ejector fork. The medicine applicator stick is disinfected and lifted up within a short few seconds. Then, the controller 11 controls the electric motor 12 to rotate reversely to drop down the lifting frame 14, so as to complete a working cycle.

In this embodiment, the medicine applicator stick bin 1, the lifting mechanism, the access window 4 and the box body 19 are used to enable the device have multiple dustproof, exposure-proof, and touch-proof function. So, during the process of taking out the medicine applicator stick for use, various pollution routes are eliminated by ultraviolet disinfection on this stick and by automatically taking out this stick. In addition, the box body 19 is provided with the charging port, the electric quantity indicator light and the charging indicator light, which facilitates the replenishment of the electric quantity of the device.

Specific embodiments are used in this specification for illustration of the principles and implementations of the present disclosure. The description of the above embodiments is merely used to help understand the method and core concept of the present disclosure. In addition, those of ordinary skill in the art may make modifications to the specific implementations and application scope in accordance with the concept of the present disclosure. In conclusion, the content of this specification should not be construed as a limitation to the present disclosure.

1. A device for automatically taking out one of medicine applicator sticks for use, the device comprising a box body and a medicine applicator stick bin, a lifting mechanism, a disinfection mechanism and a controller which are provided in the box body, wherein the medicine applicator stick bin is configured for accommodating the medicine applicator sticks and capable of sliding down the one of the medicine applicator sticks at a time, and the medicine applicator stick bin is jointed to an ejector fork of the lifting mechanism via an inclined sliding track of the box body, the lifting mechanism is capable of driving the one of the medicine applicator sticks to rise, the disinfection mechanism is arranged to correspond to a dipping end of the one of the medicine applicator sticks and is capable of disinfecting the dipping end of the one of the medicine applicator sticks, the lifting mechanism and the disinfection mechanism are each electrically connected to the controller.

2. The device for automatically taking out one of medicine applicator sticks for use according to claim 1, wherein a bin cover of the medicine applicator stick bin is snap-fitted with a bin body of the medicine applicator stick bin, the bin body is snap-fitted with the box body, a bottom of the medicine applicator stick bin is an inclined surface; a discharge port is provided on a bin wall, which intersects with the inclined surface, of the medicine applicator stick bin; and a sliding cover is slidably provided on the discharge port.

3. The device for automatically taking out one of medicine applicator sticks for use according to claim 2, wherein a side of the medicine applicator stick bin is provided with an access window; a side wall of the access window is provided with bosses which are configured to abut against a bottom of the sliding cover; the access window is hollow and a top cover is rotatably provided at a top of the access window; and the top cover is flush with the bin cover.

4. The device for automatically taking out one of medicine applicator sticks for use according to claim 3, wherein the disinfection mechanism comprises an ultraviolet lamp and a grating sensor, the ultraviolet lamp and the grating sensor are each electrically connected to the controller, and the ultraviolet lamp is arranged at a bottom of the access window and corresponds to the dipping end of the one of the medicine applicator sticks.

5. The device for automatically taking out one of medicine applicator sticks for use according to claim 4, wherein the ultraviolet lamp is arranged in the box body, a fixing plate is arranged in the box body, the grating sensor is provided on a middle of the fixing plate; a top of the fixing plate is provided with a light sensor which is electrically connected to a switch of the controller.

6. The device for automatically taking out one of medicine applicator sticks for use according to claim 5, wherein the lifting mechanism comprises an electric motor, a lifting frame, a gear and a rack, a rotating shaft of the electric motor is connected to the gear, the rack is provided on the lifting frame, the gear is meshed with the rack, the ejector fork is provided at a top of the lifting frame, vertical blocking bars are provided on sides of the lifting frame, and each of the blocking bars is provided with a positioning notch, the blocking bars match with the grating sensor.

7. The device for automatically taking out one of medicine applicator sticks for use according to claim 6, wherein the rotating shaft of the electric motor is connected to the gear via a speed reducer.

8. The device for automatically taking out one of medicine applicator sticks for use according to claim 1, wherein a bottom of the box body is provided with a base which is provided with a power supply cabin for accommodating a power supply, an electric quantity indicator light and a charging port.

9. The device for automatically taking out one of medicine applicator sticks for use according to claim 8, wherein the base is provided with an anti-inclining means.
